(19) United States
(12) Patent Application Publication
(10) Pub. No.: US 2009/0163958 A1
(43) Pub. Date: Jun. 25, 2009

(54) COMPOSITIONS, DEVICES, SYSTEMS, AND METHODS FOR INHIBITING AN INFLAMMATORY RESPONSE

(76) Inventors: Peter Tarcha, Lake Villa, IL (US); Abhijeet Joshi, Austin, TX (US); Brian Bergeron, Austin, TX (US); Diana Martinez, Austin, TX (US)

Correspondence Address: PAUL D. YASGER;ABBOTT LABORATORIES, 100 ABBOTT PARK ROAD, DEPT. 377/AP6A, ABBOTT PARK, IL, 60064-6008, US

(21) Appl. No.: 11/961,500

(22) Filed: Dec. 20, 2007

(51) Int. Cl. A61B 17/58

(52) U.S. Cl. 606/280; 606/246

(57) ABSTRACT

The present disclosure relates, according to some embodiments, to compositions, devices, systems, and methods for inhibiting an inflammatory response (e.g., an inflammatory response associated with spinal surgery). For example, some embodiments of the present disclosure may reduce and/or eliminate an inflammatory response associated with friction between a soft tissue and an implant and/or between a soft tissue and a hard tissue.

### FIELD OF THE DISCLOSURE

The present disclosure relates, in some embodiments, to compositions, devices, systems, and methods for inhibiting an inflammatory response, for example, at or near a surgical site and/or an implant.

### BACKGROUND OF THE DISCLOSURE

The spine comprises vertebrae, intervertebral discs separating the vertebrae, the sacrum, and coccyx. Intervertebral discs comprise a tough, fibrous outer ring, called the annulus fibrosis, and a viscous, fluid-filled central core called the nucleus pulposus. In addition, the spine comprises a spinal canal that houses the spinal cord. The spinal canal is protected by the intervertebral foramen in the vertebral regions and by the ligamentum flavum and the posterior longitudinal ligament in the intervertebral spaces. The spinal cord is enclosed within meninges, which consists of three layers of connective tissue. Blood vessels and capillaries that supply blood to the spinal cord run through the meninges and the space inside the outer layer (membrane) of the meninges is filled with cerebrospinal fluid.

Spinal diseases and injuries continue to be among the most painful and dehabilitating, despite advances in the understanding of spinal physiology, neurophysiology, pathology, and trauma. Attempts to treat a spinal condition may be less effective than desired, in part, because of the unique attributes of the spinal environment. For example, the flexibility and range of motion of the spine may generate large intradiscal pressures during normal loading of the spine. These pressures may interfere with normal healing processes. Sutures in connective tissues of the spine (e.g., annulus fibrosis, meninges) may pull out quickly, may aggravate existing tears and/or may nucleate new tears. In addition, drug delivery may be difficult since areas of the spine (e.g., annulus fibrosis) lack a direct blood supply.

Inflammation may also impede a favorable surgical outcome. For example, following neck surgery (e.g., anterior cervical spine surgery), patients may have trouble swallowing (e.g., dysphagia). Dysphasia may be attended by adhesions (e.g., between esophageal structures and prevertebral fascia). Formation of these adhesions may be attributable to an inflammatory response during or after surgery.

### SUMMARY

Accordingly, a need has arisen for improved compositions, devices, systems, and methods for inhibiting an inflammatory response associated with spinal surgery (e.g., to place an implant).

The present disclosure relates, according to some embodiments, to compositions, devices, systems, and methods for inhibiting an inflammatory response (e.g., an inflammatory response associated with spinal surgery). For example, some embodiments of the present disclosure may reduce and/or eliminate an inflammatory response associated with friction between a soft tissue and an implant and/or between a soft tissue and a hard tissue.

The present disclosure relates, in some embodiments, to spinal implants. A spinal implant may comprise, for example, (a) a spinal implant body having a surface, and (b) a surface composition covering at least a portion of the surface, the surface composition comprising a biocompatible lubricant polymer and a pharmaceutically effective amount of a pharmaceutical compound. A spinal implant body may comprise, for example, a pedicle screw, a polyaxial screw, an interbody spacer, an anterior cervical plate, an open hook, a rod, a rod-to-rod connector, a cable, a cam, a bone plate, a bone screw, a vertical endplate, a cage, an artificial disc, and combinations thereof. According to some embodiments, a spinal implant may comprise a material selected from bioglass, a blood cell, a bone—allograft, a bone autograft, a bone cement, a bone chip, calcium, calcium carbonate, calcium phosphate, calcium sulfate, a ceramic, a demineralized bone, a glass, gold, a liposome, a mesenchymal cell, an osteoblast, a phosphate glass, a platelet, albumin, casein, a whey protein, a plant protein, a fish protein), a steel, a synthetic cancellous bone void filler, a thrombin, titanium, tricalcium phosphate, trimethylene carbonate (TMC), and combinations thereof.

A biocompatible lubricant polymer may comprise a material selected from a photo polymerizable semi-interpenetrating anhydride network, a crosslinked polymer network with an associated long chain, biocompatible hydrophilic polymer, a hyaluronan hydrogel (e.g., crosslinked hyaluronan-adipic dyhydrazide and/or hyaluronan-aldehyde), a poly(ortho ester), a high molecular weight dextran polymer, high density polyethylene, low density polyethylene, polytetrafluoroethylene, and combinations thereof according to some embodiments. For example, a biocompatible lubricant polymer, according to some embodiments, may consist of a photo polymerizable semi-interpenetrating anhydride network comprising a photo polymerizable monomer selected from a methacrylic anhydride of sebacic acid (SA-Me₂), a methacrylic anhydride of 1,3bis(p-carboxy phenoxy)propane (CPP-Me₂), and combinations thereof. In some embodiments, a biocompatible lubricant polymer may consist of a crosslinked polymer network and an associated long chain, biocompatible hydrophilic polymer, the crosslinked polymer network comprising a polymer selected from a polyacrylate, a polymethacrylate, a polyurethane, a polyethylene and polypropylene co-difunctional polymer, a polyvinyl chloride, a epoxide, a polyamide, a polyester and alkyd copolymer, and combinations thereof. A biocompatible lubricant polymer may consist of a crosslinked polymer network and an associated long chain, biocompatible hydrophilic polymer, the associated long chain, biocompatible hydrophilic polymer comprising a polymer selected from a poly(N-vinyl lactam, a poly(vinylpyrrolidone), a poly(ethylene oxide), a poly(propylene oxide). a polyacrylamide, a cellulosic material, methyl cellulose, a polyanhydride, a polyacrylic acid, a polyvinyl alcohol, a polyvinyl ether, and combinations thereof in some embodiments. A poly(ortho ester) may be selected from poly(ortho ester) I, poly(ortho ester) II, poly(ortho ester) III, poly(ortho ester) IV, and combinations thereof. A high molecular weight dextran polymer, according to some embodiments, may comprise dextran having a molecular weight from about 100,000 daltons to about 2,000,000 daltons.

According to some embodiments, a pharmaceutical compound may comprise a compound selected from an adhesive, an arterial vessel wall irritant, a bone morphogenic protein, an extracellular matrix component, an inflammatory cytokine, a polymer, and combinations thereof. A pharmaceutical compound may comprise a compound selected from an analgesic, an antimicrobial agent, an anti-inflammatory agent, a fibrosis-inducing agent, and combinations thereof.

A surface of a spinal implant, in some embodiments, may be from about 0.5 millimeters to about 20 millimeters in its longest dimension. According to some embodiments, a surface of a spinal implant may have a surface roughness (Ra) of from about 25 to about 50 measured without the surface composition. For example, a spinal implant may have a surface roughness (Ra) of from about 30 to about 45 measured with the surface composition. A spinal implant surface may have a coefficient of friction of from about 0.001 to about 0.2 measured with the surface composition and/or may have a thickness of from about 0.1 microns to about 1 millimeter in some embodiments.

The present disclosure also relates, in some embodiments, to methods of reducing and/or eliminating an inflammatory response. A method may include, for example, contacting at least a portion of a subject's spine with a spinal implant comprising a spinal implant body having a surface, and a surface composition covering at least a portion of the surface, the surface composition comprising a biocompatible lubricant polymer and a pharmaceutically effective amount of a pharmaceutical compound.

A spinal implant body for reducing and/or eliminating an inflammatory response may comprise, for example, a pedicle screw, a polyaxial screw, an interbody spacer, an anterior cervical plate, an open hook, a rod, a rod-to-rod connector, a cable, a cam, a bone plate, a bone screw, a vertical endplate, a cage, an artificial disc, and combinations thereof. According to some embodiments, a spinal implant for reducing and/or eliminating an inflammatory response may comprise a material selected from bioglass, a blood cell, a bone—allograft, a bone autograft, a bone cement, a bone chip, calcium, calcium carbonate, calcium phosphate, calcium sulfate, a ceramic, a demineralized bone, a glass, gold, a liposome, a mesenchymal cell, an osteoblast, a phosphate glass, a platelet, albumin, casein, a whey protein, a plant protein, a fish protein), a steel, a synthetic cancellous bone void filler, a thrombin, titanium, tricalcium phosphate, trimethylene carbonate (TMC), and combinations thereof. A biocompatible lubricant polymer may comprise a material selected from a photo polymerizable semi-interpenetrating anhydride network, a crosslinked polymer network with an associated long chain, biocompatible hydrophilic polymer, a hyaluronan hydrogel (e.g., crosslinked hyaluronan-adipic dyhydrazide and/or hyaluronan-aldehyde), a poly(ortho ester), a high molecular weight dextran polymer, high density polyethylene, low density polyethylene, polytetrafluoroethylene, and combinations thereof according to some embodiments.

According to some embodiments, a pharmaceutical compound for use in an implant for reducing and/or eliminating an inflammatory response may comprise a compound selected from an adhesive, an arterial vessel wall irritant, a bone morphogenic protein, an extracellular matrix component, an inflammatory cytokine, a polymer, and combinations thereof. A pharmaceutical compound may comprise a compound selected from an analgesic, an antimicrobial agent, an anti-inflammatory agent, a fibrosis-inducing agent, and combinations thereof.

A surface of a spinal implant for reducing and/or eliminating an inflammatory response, in some embodiments, may be from about 0.5 millimeters to about 20 millimeters in its longest dimension. According to some embodiments, a surface of a spinal implant may have a surface roughness (Ra) of from about 25 to about 50 measured without the surface composition. For example, a spinal implant may have a surface roughness (Ra) of from about 30 to about 45 measured with the surface composition. A spinal implant surface may have a coefficient of friction of from about 0.001 to about 0.2 measured with the surface composition and/or may have a thickness of from about 0.1 microns to about 1 millimeter in some embodiments.

In some embodiments, the present disclosure relates to methods of manufacturing a spinal implant. For example, a method may comprise (a) providing a spinal implant body having a smooth surface, and (b) depositing on the smooth surface a surface composition comprising a biocompatible lubricant polymer and a pharmaceutical compound, wherein the surface composition is configured and arranged to deliver a pharmaceutically effective amount of the pharmaceutical compound to the subject's spine. Depositing, in some embodiments, may comprise uniformly depositing the surface composition on to the smooth surface. According to some embodiments, depositing may comprise applying, loading, coating, covering, injecting, spraying, dipping, printing and/or jetting the surface composition on to the smooth surface. Printing may comprise (a) providing a fluid-dispenser having a dispensing element operable to dispense the surface composition in discrete droplets, wherein each droplet has a controlled trajectory, (b) creating relative movement between the dispensing element and the implant (e.g., prosthesis) to define a dispensing path, and (c) selectively dispensing the surface composition from the fluid-dispenser in a raster format to a predetermined portion of the spinal implant surface along the dispensing path in some embodiments. A manufacturing method may comprise, in some embodiments, depositing a sufficient amount of the surface composition to give from about 1 microgram to about 100 micrograms of the pharmaceutical agent per millimeter of the surface. According to some embodiments, a manufacturing method may comprise curing a surface composition. Curing may comprise, for example, hydrating, heating, or irradiating the surface composition. A manufacturing method may comprise sterilizing and/or sanitizing a spinal implant.

The present disclosure also relates to systems for reducing and/or eliminating an inflammatory response in some embodiments. For example, a system for reducing and/or eliminating an inflammatory response may comprise (a) a first spinal implant comprising a spinal implant body having a first surface, and a first surface composition covering at least a portion of the first surface, the first surface composition comprising a first biocompatible lubricant polymer and a pharmaceutically effective amount of a first pharmaceutical compound, and (b) a second spinal implant. In some embodiments, the first and second spinal implants may be the same or different. A second spinal implany may comprise, according to some embodiments, a spinal implant body having a second surface, and a second surface composition covering at least a portion of the second surface, the second surface composition comprising a second biocompatible lubricant polymer and a pharmaceutically effective amount of a second pharmaceutical compound.

According to some embodiments, a system for reducing and/or eliminating an inflammatory response may comprise (a) a pharmaceutical composition comprising a pharmaceutically effective amount of a first pharmaceutical agent and a carrier, and (b) a spinal implant comprising a spinal implant body having a surface, and a surface composition covering at least a portion of the surface, the surface composition comprising a biocompatible lubricant polymer and a pharmaceutically effective amount of a second pharmaceutical compound. A pharmaceutical composition for use in a system for reducing and/or eliminating an inflammatory response may be configured and arranged for intravenous delivery, intramuscular delivery, oral delivery, or transdermal delivery to subject. A first and second pharmaceutical agent, in some embodiments, may be the same or different.

The present disclosure provides, in some embodiments, implantable bone screws. For example, an implantable bone screw may comprise (a) an implantable bone screw body having bone screw threads and a bone screw head having a surface, and (b) a surface composition covering at least a portion of the surface, the surface composition comprising a biocompatible lubricant polymer and a pharmaceutically effective amount of a pharmaceutical compound. A bone screw may be configured and arranged as a pedicle screw or a polyaxial screw, according to some embodiments. The present disclosure also provides, in some embodiments, implantable bone plates. For example, an implantable bone plate may comprise (a) an implantable bone plate body having a surface, and (b) a surface composition covering at least a portion of the surface, the surface composition comprising a biocompatible lubricant polymer and a pharmaceutically effective amount of a pharmaceutical compound. A bone plate may be configured and arranged as a cervical plate, according to some embodiments.

### BRIEF DESCRIPTION OF THE DRAWINGS

Some embodiments of the disclosure may be understood by referring, in part, to the present disclosure and the accompanying drawing, wherein:

FIG. 1 illustrates a perspective view of an embodiment of a cervical base plate for a bone plate system according to a specific example embodiment of the disclosure.

### DETAILED DESCRIPTION

The present disclosure relates, in some embodiments, to compositions, devices, systems, and methods to reduce and/or minimize an inflammatory response and/or fibrosis (e.g., a spinal inflammatory response and/or fibrosis). Without limiting the scope of any particular embodiment, the extent of an inflammatory response may be related (e.g., linearly related and/or non-linearly) to the extent of friction between a surface (e.g., an implant surface and/or a bone surface) and a tissue (e.g., soft tissue).

### Implant

According to some embodiments, an implant (e.g., a spinal implant) may be configured and arranged to reduce and/or eliminate an inflammatory response. An implant surface may comprise, according to some embodiments, any chemical, physical, and/or physiochemical modification that alone or in part reduces and/or eliminates an inflammatory response. A modification may include, for example, changing one or more surface chemical properties (e.g., nonfouling surfaces that resist adhesion of cells and protein adsorption, making the surface(s) hydrophilic or hydrophobic, altering surface charge density, radiation grafting of hydrogels, using interpenetrating polymeric networks, and the like). For example, an implant surface may be configured and arranged to reduce and/or eliminate friction (e.g., binding, rubbing, abrading, eroding, fouling, and the like) between the implant and nearby tissue (e.g., soft tissue). For example, an implant surface may be formed or fashioned to be smooth in some embodiments. An implant surface may be configured and arranged to be smooth, for example, by minimizing and/or excluding rough surfaces, edges, corners, protrusions, and the like. An implant surface may be configured and arranged to be smooth by including a surface composition (e.g., a coating) that is smooth and/or becomes smooth when desired (e.g., upon contact with radiation and/or water). A surface composition may include material sufficient to make a surface slippery (e.g., lower the coefficient of friction). A surface composition (e.g., a coating) may comprise, for example, a biocompatible polymer (e.g., a lubricant polymer), an anti-inflammatory agent, and/or an anti-fibrotic agent.

A spinal implant, according to some embodiments, may comprise any implant that supports, augments, and/or replaces (e.g., fully or partially) one or more spinal structures. In some embodiments, a spinal implant may be situated in contact with and/or near a spinal structure. Non-limiting examples of spinal structures may include a vertebrae (e.g., vertebral end plate, vertebral foramen, vertebral body, cortical rim, cancellous, pedicle, spinous process, lamina, superior articular process, transverse process), an intervertebral disc, a sacrum, a coccyx, an annulus fibrosis, a nucleus pulposus, a spinal canal a spinal cord, a ligamentum flavum, a posterior longitudinal ligament, a meninx (e.g., dura mater, arachnoid mater, pia mater), cerebrospinal fluid and/or any portion thereof. A spinal implant, according to some embodiments of the disclosure, may include all or a portion of a pedicle fixation system (e.g., a pedicle screw), a transforaminal lumbar interbody spacer, a thoracolumbar fixation system (e.g., a polyaxial screw), a thoracolumbar fixation system, (e.g., a pedicle screw), a posterior thoracolumbar fixation system, a transverse connector posterior spine implant (e.g., a pedicle screw), an anterior cervical plate system, a cervicothoracic fixation system (e.g., a polyaxial screw, an open hook, a rod, and/or a rod-to-rod connector), an occipital cervical fixation system, a cable fixation system (e.g., a cable and/or a cam), a bone plate system (e.g., a titanium bone plate and/or screw), an anterior lumbar system (e.g., a vertical endplate), a cage, an artificial disc, and/or an interbody spacer (e.g., a vertebral interbody spacer, a posterior lumbar interbody spacer and/or an expandable lumbar interbody spacer).

In some embodiments, an implant includes a surface that is physically smooth. For example, surface roughness may be measured as set forth, for example, in ASME B46.1 (2002). For example, surface roughness may be expressed as the average deviation of the profile height of all features from the mean line (Rₐ). The surface roughness of an implant, according to some embodiments, may be from about 25 Ra to about 50 Ra (e.g., about 30 Ra to about 45 Ra) as measured with or without a surface composition. A physically smooth surface may be associated with a lower coefficient of friction compared to a device having a rougher surface.

In some embodiments, an implant comprises a surface that is coated with a surface composition. A surface composition may reduce friction across a coated surface. For example, a surface composition may reduce the coefficient of friction by any amount that effectively reduces an associated inflammatory response. A coefficient of friction of device having a surface composition may be about 0.01%, about 0.05%, about 0.1%, about 0.5%, about 1%, about 10%, about 25 %, about 35%, about 40%, about 50 %, about 60%, about 65%, about 75%, about 90%, and/or about 99% of the coefficient of friction of a device (e.g., the same device) without a surface composition. A coefficient of friction of a surface including a surface composition may be from about 0.001 to about 0.2.

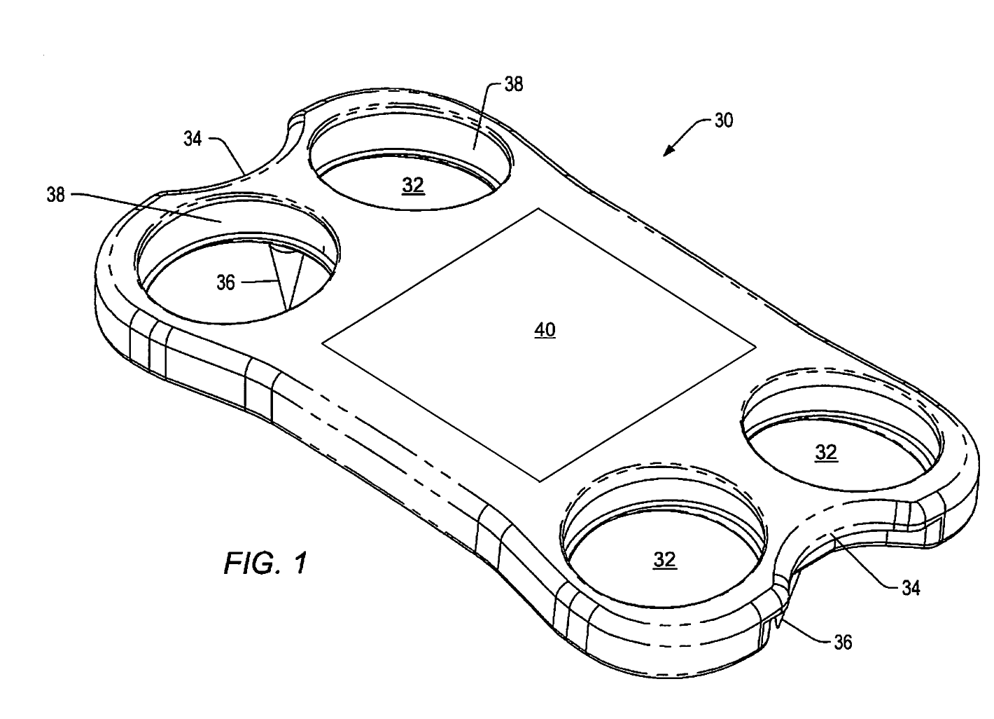

A cervical base plate according to a specific example embodiment of the disclosure is illustrated in FIG. 1. As shown, cervical base plate 30 may include openings 32, indentions 34, and spikes 36. Opening 32 may define wall 38 of base plate 30. Cervical base plate 30 may further include surface coating 40 on a portion of its surface as shown.

### Compositions-Matrixes

According to some embodiments, the present disclosure relates to surface compositions configured and arranged to reduce and/or eliminate an inflammatory response (e.g., an inflammatory response associated with friction between a spinal implant and a soft tissue). A surface composition may comprise, according to some embodiments, a lubricant polymer. A surface composition may include, according to some embodiments of the disclosure, a product of a polymerization reaction inclusive of homopolymers, copolymers, terpolymers, and the like, whether natural or synthetic, including random, alternating, block, graft, branched, cross-linked, blends, compositions of blends and variations thereof. A polymer (e.g., a lubricant polymer) may be in true solution, saturated, or suspended as particles or supersaturated in the surface composition. A polymer (e.g., a lubricant polymer) may be biocompatible and/or biodegradable.

In some embodiments, a surface composition may include a lubricant polymer selected from the group consisting of alginate, aliphatic polyesters, carbohydrates, cellulose, cellulose derivatives (e.g., HPC), chitin, chitosan, chitosan derivatives, collagen, collagen—native fibrous, collagen—recombinant derived, collagen—reconstituted fibrous, collagen—soluble, collagen—Types 1 to 20, copolymers of glycolide, copolymers of lactide, cyanoacrylate, dacron, elastin, felt, fibrin, gelatin, glycolide/1-lactide copolymers (PGA/PLLA), glycolide/trimethylene carbonate copolymers (PGA/TMC), glycosaminoglycans, hyaluronic acid, hyaluronic acid derivatives, hydrogel, hydroxyethyl methacrylate, lactide/ε-caprolactone copolymers, lactide/σ-valerolactone copolymers, lactide/tetramethylglycolide copolymers, lactide/trimethylene carbonate copolymers, 1-lactide/dl-lactide copolymers, polymethyl methacrylate (PMMA), polymethyl methacrylate-N-vinyl pyrrolidone copolymers, polymethyl methacrylate-styrene (MMA-styrene), nitinol, nylon-2, oligoethylenimine (OEI), OEI-HD (e.g., a condensation product of OEI with hexanedioldiacrylate), oxidized regenerated cellulose, PHBA/γ-hydroxyvalerate copolymers (PHBA/HVA), PLA/polyethylene oxide copolymers, PLA-polyethylene oxide (PELA), polyethylenimine (PEI), poly(amino acids), poly(trimethylene carbonates), poly hydroxyalkanoate polymers (PHA), poly(alklyene oxalates), poly(butylene diglycolate), poly(glycerol sebacate), poly(hydroxy butyrate) (PHB), poly(methacrylic acid), poly(n-vinyl pyrrolidone), poly(ortho esters), poly(styrene sulfonate), poly-β-alkanoic acids, poly-β-hydroxybutyrate (PBA), poly-β-hydroxypropionate (PHPA), poly-β-malic acid (PMLA), poly-ε-caprolactone (PCL), poly-σ-valerolactone, polyalkyl-2-cyanoacrylates, polyanhydrides, polycyanoacrylates, polydepsipeptides, polydihydropyrans, poly-DL-lactide (PDLLA), polyester, polyesteramides, polyester-polyalkylene oxide block copolymers, polyesters of oxalic acid, polyethylene glycol-crosslinked, polyethylene glycol-poly(vinyl PEG), polyethylene glycol (PEG), polyethylene oxide, polyglycan esters, polyglycolide (PGA), polyiminocarbonates, polylactides (PLA), poly-1-lactide (PLLA), polymethyl methacrylate (PMMA), polyorthoesters, poly-p-dioxanone (PDO), polypeptides, polyphosphazenes, polysaccharides, polyurethanes (PU), polyvinyl alcohol (PVA), pseudo-poly(amino acids), radiopacifiers, salts, silicone, silk, starch, synthetic polymers, tyrosine based polymers (e.g., high density polyethylene, low density polyethylene, polytetrafluoroethylene), and combinations thereof. An implant may include a material selected from the group consisting of bioglass, blood cells, bone—allograft or autograft, bone cement, bone chips, calcium, calcium carbonate, calcium phosphate, calcium sulfate, ceramics, demineralized bone, glass, gold, graphite, carbon derived materials, nano-manufactured materials such as single and double walled nanotubes, liposomes, mesenchymal cells, osteoblasts, phosphate glasses, platelets, proteins (e.g., albumin, casein, whey proteins, plant proteins, and fish proteins), proteins modified, steel (e.g. stainless steel), synthetic cancellous bone void fillers, thrombin, titanium, tricalcium phosphate, trimethylene carbonate (TMC), and combinations thereof.

A lubricant polymer may include, for example, a photo polymerizable semi-interpenetrating anhydride network. Examples of photo polymerizable monomers include, without limitation, a methacrylic anhydride of sebacic acid (SA-Me₂) and/or a methacrylic anhydride of 1,3bis(p-carboxy phenoxy)propane (CPP-Me₂). One or more photo polymerizable monomers may be mixed with an initiator (e.g., an ultraviolet initiator). A mixture of monomer(s) and initiator may be cured upon exposure to light (e.g., UV light). A mixture of monomer(s) and initiator may be applied to an implant surface in cured or uncured form.

A lubricant polymer may comprise, according to some embodiments, a crosslinked polymer network with (e.g., containing) an associated long chain, biocompatible, hydrophilic polymer (e.g., a LubriLAST™ Lubricous Coating). Examples of functional groups that may be crosslinked to form a polymer network include, without limitation, amino groups, hydroxyl groups, amido groups, carboxylic acid groups and derivatives thereof, sulfhydryl (SH) groups, unsaturated carbon bond and heteroatom bonds, N—COOH groups, N(C═O)H S(OR) groups, alkyd/dry resins, formaldehyde condensates, methylol acrylamides, and/or allylic groups. Examples of a polymer in a polymer network may include, without limitation, polyacrylates, polymethacrylates, polyurethanes, polyethylene and polypropylene co-difunctional polymers, polyvinyl chlorides, epoxides, polyamides, polyesters and alkyd copolymers. The hydrophilic polymer may be selected from the group consisting of poly(N-vinyl lactams, poly(vinylpyrrolidone), poly(ethylene oxide), poly(propylene oxide), polyacrylamides, cellulosics, methyl cellulose, polyanhydrides, polyacrylic acids, polyvinyl alcohols, and/or polyvinyl ethers. In addition, a crosslinked polymer network may include a crosslinking agent (e.g., an aziridine, a polyfunctional carbodiimide, a polyfunctional epoxide, an unsaturated carbon and a heteroatom bond, a melamine/urea condensate, and/or an ionic complexing agent). A crosslinked polymer network may comprise polyurethane and polyvinylpyrrolidone (PVP), wherein the PVP may facilitate formation of a swollen, gel-like layer upon contact with water.

A lubricant polymer may comprise, according to some embodiments, a hyaluronan hydrogel. For example, a hyaluronan hydrogel may be prepared by contacting a hyaluronan-adipic dyhydrazide with a hyaluronan-aldehyde. In some embodiments, a lubricant polymer may comprise a poly(ortho ester) (POE) (e.g., POE I, POE II, POE III, and/or POE IV). A POE may be included as a crosslinked polymer and/or a linear polymer. According to some embodiments, a lubricant polymer may comprise a high molecular weight dextran polymer (e.g., from about 100,000 daltons to about 2,000,000 daltons).

In some embodiments, a surface composition may comprise one or more additives selected from the group consisting of co-solvents, plasticizers, antifoaming agents, anticrater agents, coalescing solvents, bioactive agents, antimicrobial agents, antithrombogenic agents, antibiotics, pigments, paint additives, radiopacifiers and ion conductors.

According to some embodiments, a surface composition may include a pharmaceutical agent. A coating, in some embodiments, may include a uniform matrix of therapeutic agent and polymer, binder, and/or carrier. For example, as desired or necessary, a surface composition may include a pharmaceutical agent and/or a binder to carry, load, and/or release (e.g., sustained release) the agent, such as but not limited to a suitable polymer or similar carrier. According to some embodiments of the disclosure, a polymer may include a product of a polymerization reaction inclusive of homopolymers, copolymers, terpolymers, etc., whether natural or synthetic, including random, alternating, block, graft, branched, cross-linked, blends, compositions of blends and variations thereof. A polymer may be in true solution, saturated, or suspended as particles or supersaturated in the therapeutic agent. A polymer may be biocompatible and/or biodegradable.

A polymeric material (e.g., a lubricant polymer) may include a phosphorylcholine linked macromolecule in some embodiments (a “PC polymer”). For example, a polymeric material may include a macromolecule containing pendant phosphorylcholine groups such as poly(MPC_{w}:LMAₓ:HPMA_{y}:TSMA_{z}) where MPC is 2-methacryoyloxyethylphosphorylcholine, LMA is lauryl methacrylate, HPMA is hydroxypropyl methacrylate and TSMA is trimethoxysilylpropyl methacrylate, and w, x, y, and z are molar ratios of the monomers used in the feed. These values may be 23, 47, 25, and 5, respectively, but they are not necessarily the ratios that exist in the finished polymer.

A PC polymer may include, for example, 5% pendant trimethoxysilane groups, which may be used to crosslink the polymer after it is coated on a surface. These groups may also be used to chemically bond the material to a device having an appropriate surface chemistry. For example, where an implant that includes a Dacron mesh, the surface of the polyester may be hydrolyzed producing hydroxyl groups for reaction with trimethoxy silane. Alternatively, the Dacron may be formulated with impregnated fiber glass or glass powder. The glass may be the source of surface hydroxyl groups; however, it may change the mechanical properties of the Dacron.

A surface composition may be present in an active and/or inactive form. For example, a surface composition may be present in an unhydrated, unpolymerized, and/or uncured form that has little or no favorable or desirable effect on surface slip. Upon activation, a surface composition may reduce the coefficient of friction.

A surface composition may be configured and arranged to remain in contact with a spinal implant surface for over about 1 day, up to about 2 days, up to about 3 days, up to about 1 week, up to about 2 weeks, up to about 3 weeks, up to about 4 weeks, up to about 1 month, up to about 2 months, up to about 3 months, and/or up to about 6 months or longer. The durability of a surface composition may be related to and/or adjusted by the composition, the thickness, and/or the environment in which it is placed.

### Compositions—Drugs

An implant, according to some embodiments, may include a composition to elicit and/or impede a specific biologic response. In some embodiments, an implant may include a composition formulated to reduce and/or eliminate one or more aspects of an inflammatory response according to some embodiments. For example, an implant may include a releasable pharmaceutical agent that enhances or impedes fibrosis. A pharmaceutical agent may include, for example, an anti-inflammatory agent, and/or an anti-adhesive agent. An implant, in some embodiments, may include an anti-adhesion compound (e.g., on a portion of an implant that may contact a nerve root to minimize or avoid painful tethering of scar tissue to a nerve root).

According to some embodiments, a composition including a pharmaceutical agent may be carried on and/or eluted by at least a portion of an implant. Thus, an implant may have one or more portions that include a therapeutic composition and one or more portions that lack a therapeutic composition. For example, an implant may have a domain or domains configured and arranged to confer structure (e.g., shape, rigidity, resilience, etc.) and a domain or domains containing a pharmaceutical agent (e.g., layers, wells, and/or coatings).

A pharmaceutical agent may be included in a pharmaceutical agent elution matrix that is configured and arranged to release the pharmaceutical agent upon implantation. A spinal implant may further include a coating on at least a potion of the spinal implant, the coating comprising the pharmaceutical agent elution matrix. An implant, in some embodiments, may include a first surface and a second surface. A first surface may be configured and arranged to face tissue (e.g., soft tissue) upon implantation. A second surface may be configured and arranged to face a hard material (e.g., bone and/or implant) upon implantation. A first surface and/or a second surface may comprise a pharmaceutical agent elution matrix. In some embodiments, an implant may include a biocompatible material and/or a biodegradable material. A spinal implant may include polyester, polytetrafluoroethylene, or polyester and/or polytetrafluoroethylene in some embodiments. A spinal implant may include a polymer selected from the group consisting of a phosphorylcholine linked macromolecule, an oligoethylenimine, and a polyethylenimine.

A pharmaceutical agent, in some embodiments, may be selected from the group consisting of an analgesic, an antimicrobial agent, an anti-inflammatory agent, a fibrosis-inducing agent (e.g., an adhesive, an arterial vessel wall irritant, a bone morphogenic protein, an extracellular matrix component, an inflammatory cytokine, a polymer, and combinations thereof), and combinations thereof. A fibrosis-inducing agent may be selected from the group consisting of crosslinked poly(ethylene glycol)-methylated collagen, a cyanoacrylate, a crystalline silicate, copper, ethanol, metallic beryllium, an oxide of metallic beryllium, neomycin, quartz dust, silica, silk, talc, talcum powder, wool, bleomycin, bone morphogenic protein-2, bone morphogenic protein-3, bone morphogenic protein-4, bone morphogenic protein-5, bone morphogenic protein-6, bone morphogenic protein-7, connective tissue growth factor, collagen, fibrin, fibrinogen, fibronectin, basic fibroblast growth factor, granulocyte-macrophage colony stimulating factor, growth hormones, insulin growth factor-1, interleukin-1, interleukin-6, interleukin-8, nerve growth factor, platelet-derived growth factor, transforming growth factor-beta, tumor necrosis factor alpha, vascular endothelial growth factor, leptin, chitosan, N-carboxybutylchitosan, a poly(alkylcyanoacrylate), poly(ethylene-co-vinylacetate), poly(ethylene terephthalate), a polylysine, polytetrafluoroethylene, a polyurethane, an RGD protein, vinyl chloride, and combinations thereof.

A pharmaceutical agent suitable for inclusion in an implant of the disclosure, in some embodiments, may include a protein (e.g., peptide (e.g., adhesion peptide), enzyme, antibody, receptor, receptor ligand), a carbohydrate (e.g., monosaccharide, disaccharide, polysaccharide (linear or branched)), a lipid (e.g., prostaglandin, eicosanoid, steroid), a nucleic acid (e.g., DNA, RNA, siRNA, microRNA, ribozyme, virus, vector, coding sequence, antisense sequence, nucleotide), and/or combinations thereof. In some embodiments, a pharmaceutical agent may include one or more of the compounds listed in TABLE 1 and/or analogues and derivatives thereof. For example, a pharmaceutical agent may include alpha-interferon, an amino acid, an angiogenic agent, an anti-allergic agent, an anti-angiogenic agent, an antiarrhythmic agent, an antibiotic, an anti-coagulant agent, an anti-fibrin agent, an anti-fungal agent, an anti-inflammatory agent, an anti-neoplastic agent, an antioxidant an anti-platelet agent, an anti-proliferative agent, an anti-rejection agent, an anti-thrombonic agent, an anti-viral drug, bioactive RGD, a blood clotting agent, a cell, a chemotherapeutic agent, a fibrosis-inducing agent, a fibrosis-inhibiting agent, a growth factor, a hormone, a nitric oxide or a nitric oxide donor, nitroprusside, a phosphodiesterase inhibitor, a proliferative agent, a prostaglandin inhibitor, a proteoglycan, a radioactive material, a serotonin blocker, a super oxide dismutase, a super oxide dismutase mimetic, suramin, a thioprotease inhibitor, triazolopyrimidine, a tyrosine kinase inhibitor, a vasodilator, and/or a vitamin. In some embodiments, a pharmaceutical agent may include a compound selected from the group consisting of 1-α-25 dihydroxyvitamin D₃, alcohol, all-trans retinoic acid (ATRA), angiotensin II antagonists, anti-tumor necrosis factor, beta-blocker, carcinogens, chondroitin, clopidegrel, collagen inhibitors, colony stimulating factors, coumadin, cyclosporine A, cytokines, dentin, diethylstibesterol, etretinate, glucosamine, glycosaminoglycans, growth factor antagonists or inhibitors, heparin sulfate proteoglycan, immoxidal, immune modulator agents (e.g., immunosuppressant agents), inflammatory mediator, insulin, isotretinoin (13-cis retinoic acid), lipid lowering agents (e.g., cholesterol reducers, HMC-CoA reductase inhibitors (statins)), lysine (e.g., polylysine), methylation inhibitors, N[G]-nitro-L-arginine methyl ester (L-NAME), plavix, polyphenol, PR39, prednisone, signal transduction factors, signaling proteins, somatomedins, thrombin, thrombin inhibitor, ticlid, and combinations thereof.

TABLE 1
Pharmaceutical Agents

```
alpha-interferon
amino acid
    L-arginine
analgesic
    acetaminophen
    aspirin
    codeine
    cox2 inhibitor
    ibuprofen
    morphine
    naproxin
    nonsteroidal anti-inflammatory drug
angiogenic agent
    angiogenin
    angiotropin
    bone morphogenic protein (BMP)
    epidermal growth factor (EGF)
    fibrin
    fibroblast growth factor - acidic (aFGF) and basic (bFGF)
    granulocyte-macrophage colony stimulating factor (GM-CSF)
    hepatocyte growth factor (HGF)
    hypoxia-inducible factor-1 (HIF-1)
    indian hedgehog (inh)
    insulin growth factor-1 (IGF-1)
    interleukin-8 (IL-8)
    macrophage antigen 1 (Mac-1)
    nicotinamide
    platelet-derived endothelial cell growth factor (PD-ECGF)
    platelet-derived growth factor (PDGF)
    transforming growth factors α (TGF-α) & β (TGF-β)
    tumor necrosis factor-α (TNF-α)
    vascular endothelial growth factor (VEGF)
    vascular permeability factor (VPF)
anti-allergic agent
    permirolast potassium
antiarrhythmic agent
    amiodarone
    diltiazem
    lidocaine
    procainamide
    sotalol
antibiotic
    cipro
    erythromycin
    flagyl
    imipenem
    penicillin
    vancomycin
    zosyn
anti-coagulant agent
    heparin
    lovenox
anti-fibrin agent
anti-fungal agent
anti-inflammatory agent
    aspirin
    clobetasol
    colchicine
    dexamethasone
    glucocorticoid
        betamethasone
        budesonide
        cortisone
        dexamethasone
        hydrocortisone
        methylprednisolone
        prednisolone
    non-steroidal anti-inflammatory agent
        acetominophen
        diclofenac
        diclofenac
        diflunisal
        etodolac
        fenoprofen
        flurbiprofen
        ibuprofen
                indomethacin
        ketoprofen
        ketorolac
        meclofenamic acid
        naproxen
        phenylbutazone
        piroxicam
        sulindac
    tacrolimus
anti-neoplastic agent
    alkylating agent
        altretamine
        bendamucine
        carboplatin
        carmustine
        cisplatin
        cyclophosphamide
        fotemustine
        ifosfamide
        lomustine
        nimustine
        prednimustine
        treosulfin
    antibiotic
        doxorubicin hydrochloride
        mitomycin
    antimetabolite
        azathioprine
        fluorouracil
        gemcitabine
        mercaptopurine
        methotrexate
        pentostatin
        trimetrexate
    antimitotic agent
        docetaxel
        paclitaxel
        vinblastine
        vincristine
    ceramide
    estradiol (e.g., 17-β-estradiol)
    flutamide
    imatinib
    levamisole
    oxaliplatin
    tamoxifen
    taxol
    topotecan
antioxidant agent
anti-platelet agent
    eptifibatide
    forskolin
    GP IIb/IIIa inhibitor
        L-703,081
anti-proliferative agent
    (+)-trans-4-(1-aminoethyl)-1-(4-pyridylcarbamoyl) cyclohexane
    amlodipine
    angiotensin converting enzyme inhibitor
        captopril
        cilazapril
        lisinopril
    anti-estrogen
        tamoxifen
    anti-restenosis agent
        40-O-(2-hydroxyethyl)rapamycin (everolimus)
        40-O-(2-hydroxyethyoxy)ethylrapamycin
        40-O-(3-hydroxypropyl)rapamycin
        40-O-tetrazolylrapamycin (zotarolimus, ABT-578)
        adenosine A2A receptor agonist
        pimecrolimus
        rapamycin (sirolimus)
        rapamycin analog
        tacrolimus
    azathioprine
    benidipine
    calcium channel blocker
        nifedipine
    cilnidipine
    cytostatic agent
        angiopeptin
    diltiazem and verapamil
    docetaxel
    doxorubicin hydrochloride
    fibroblast growth factor antagonists
    fish oil (omega 3-fatty acid)
    fluorouracil
    histamine antagonist
    lercanidipine
    lovastatin
    methotrexate
    mitomycin
    paclitaxel
    rho kinase inhibitor
    trifluperazine
    topoisomerase inhibitor
        etoposide
        topotecan
    vinblastine
    vincristine
anti-rejection agent
anti-thrombonic agent
    argatroban
    dextran
    dipyridamole
    D-phe-pro-arg-chloromethylketone (synthetic antithrombin)
    bivalirudin
    fondaparinux
    forskolin
    GP IIb/IIIa inhibitor
        L-703,081
        glycoprotein IIb/IIIa platelet membrane
        receptor antagonist antibody
    heparinoid
    hirudin
    low molecular weight heparin
    prostacyclin
    prostacyclin analogue
    recombinant hirudin
    sodium heparin
    thrombolytics
        urokinase
        recombinant urokinase
        pro-urokinase
        tissue plasminogen activator
        tenecteplase (TNK-tPA)
    vapiprost
anti-viral drug
bioactive RGD
blood clotting agent
    streptokinase
    tissue plasminogen activator
cell
    bacteria
    blood cell
    bone marrow
    fat cell
    genetically engineered epithelial cell
    lymphocytes
    muscle cell
    stem cell
    umbilical cord cell
    yeast
        Ziyphi fructus
fibrosis-inducing agent
    adhesive
        crosslinked poly(ethylene glycol)-methylated collagen
        cyanoacrylate
    arterial vessel wall irritant
        crystalline silicates
        copper
        ethanol
        metallic beryllium and oxides thereof
        neomycin
        quartz dust
        silica
        silk
        talc
        talcum powder
        wool
    bleomycin
    bone morphogenic protein (BMP)
        bone morphogenic protein-2
        bone morphogenic protein-3
        bone morphogenic protein-4
        bone morphogenic protein-5
        bone morphogenic protein-6
        bone morphogenic protein-7
    connective tissue growth factor (CTGF)
    extracellular matrix component
        collagen
        fibrin
        fibrinogen
        fibronectin
    inflammatory cytokine
        basic fibroblast growth factor (bFGF)
        granulocyte-macrophage colony stimulating factor (GM-CSF)
        growth hormones
        insulin growth factor-1 (IGF-1)
        interleukin-1 (IL-1)
        interleukin-6 (IL-6)
        interleukin-8 (IL-8)
        nerve growth factor (NGF)
        platelet-derived growth factor (PDGF)
        transforming growth factor-β (TGF-β)
        tumor necrosis factor-α (TNF-α)
        vascular endothelial growth factor (VEGF)
    leptin
    polymer
        chitosan
        N-carboxybutylchitosan
        a poly(alkylcyanoacrylate)
        poly(ethylene-co-vinylacetate)
        poly(ethylene terephthalate)
        a polylysine
        polytetrafluoroethylene (PTFE)
        a polyurethane
        RGD protein
    vinyl chloride (including a polymer of vinyl chloride)
growth factor
    autologous growth factor
    bovine derived cytokine
    cartilage derived growth factor (CDGF)
    endothelial cell growth factor (ECGF)
    fibroblast growth factor - acidic (aFGF) and basic (bFGF)
    hepatocyte growth factor (HGF)
    insulin growth factor-1 (IGF-1)
    insulin-like growth factor
    nerve growth factor (NGF) (including recombinant NGF)
    platelet-derived endothelial cell growth factor (PD-ECGF)
    platelet-derived growth factor (PDGF)
    tissue necrosis factor (TNF)
    tissue derived cytokine
    transforming growth factors α (TGF-α) & β (TGF-β)
    tumor necrosis factor α (TNF-α)
    vascular endothelial growth factor (VEGF)
    and/or vascular permeability factor (VPF)
hormone
    erythropoietin
nitric oxide or a nitric oxide donor
nitroprusside
nucleic acid
    DNA
    RNA
    siRNA
    microRNA
    antisense
phosphodiesterase inhibitor
prostaglandin inhibitor
proteoglycan
    perlecan
radioactive material
    iodine-125
    iodine-131
    iridium-192
    palladium-103
serotonin blocker
super oxide dismutase
super oxide dismutase mimetic
suramin
thioprotease inhibitor
triazolopyrimidine
tyrosine kinase inhibitor
    ST638
    tyrphostin 9 (AG-17)
vasodilator
    forskolin
    histamine
    nitroglycerin
vitamin
    vitamin C
    1-α-25 dihydroxyvitamin D₃
    vitamin E
```

A fibrosis-inducing agent may include, according to some embodiments, an adhesive, an arterial vessel wall irritant, bleomycin, a bone morphogenic protein (BMP), connective tissue growth factor (CTGF), an extracellular matrix component, an inflammatory cytokine, leptin, a polymer, and/or vinyl chloride (including a polymer of vinyl chloride). In some embodiments, a fibrosis-inducing agent may include analogues and/or derivatives of the foregoing compounds. An adhesive may include, for example, crosslinked poly(ethylene glycol)-methylated collagen and/or cyanoacrylates. An arterial vessel wall irritant may include, for example, crystalline silicates, copper, ethanol, metallic beryllium and oxides thereof, neomycin, quartz dust, silica, silk, talc, talcum powder, and/or wool. A bone morphogenic protein (BMP) may include, for example, bone morphogenic protein-2, bone morphogenic protein-3, bone morphogenic protein-4, bone morphogenic protein-5, bone morphogenic protein-6, and/or bone morphogenic protein-7. An extracellular matrix component may include, for example, collagen, fibrin, fibrinogen, and/or fibronectin. An inflammatory cytokine may include, for example, basic fibroblast growth factor (bFGF), granulocyte-macrophage colony stimulating factor (GM-CSF), growth hormones, insulin growth factor-1(IGF-1), interleukin-1(IL-1), interleukin-6(IL-6), interleukin-8(IL-8), nerve growth factor (NGF) platelet-derived growth factor (PDGF), transforming growth factor-β (TGF-β), tumor necrosis factor α (TNF-α), and/or vascular endothelial growth factor (VEGF). A polymer may include, for example, chitosan, N-carboxybutylchitosan, a poly(alkylcyanoacrylate), poly(ethylene-co-vinylacetate), poly(ethylene terephthalate), a polylysine, polytetrafluoroethylene (PTFE), a polyurethane, and/or an RGD protein.

A pharmaceutical agent, in some embodiments, may include any compound, mixture of compounds, or composition of matter consisting of a compound, which produces a therapeutic or useful result in at least one subject. A pharmaceutical agent may include a polymer, a marker, such as a radiopaque dye or particles, or may include a drug, including pharmaceutical and therapeutic agents, or an agent including inorganic or organic drugs without limitation. According to some embodiments, a pharmaceutical agent may be in various forms such as an uncharged molecule, a component of a molecular complex, and/or a pharmacologically acceptable salt (e.g., hydrochloride, hydrobromide, sulfate, laurate, palmitate, phosphate, nitrate, borate, acetate, maleate, tartrate, oleate, and salicylate).

In some embodiments, a water insoluble pharmaceutical agent may be included in an implant of the disclosure. In other embodiments, a water-soluble derivative of a water insoluble pharmaceutical agent may be included in an implant (e.g., to effectively serve as a solute). Once in a subject's body, a water-soluble derivative of a water insoluble pharmaceutical agent may be converted (e.g., by enzymes, hydrolyzed by body pH, or metabolic processes) to a biologically active form. Additionally, a pharmaceutical agent formulation may include various known forms such as solutions, dispersions, pastes, particles, granules, emulsions, suspensions and powders. The drug or agent may or may not be mixed with polymer or a solvent as desired.

A pharmaceutical agent, in some embodiments, may include a solvent. A solvent may be any single solvent or a combination of solvents. For example, a solvent may include water, aliphatic hydrocarbons, aromatic hydrocarbons, alcohols, ketones, dimethyl sulfoxide, tetrahydrofuran, dihydrofuran, dimethylacetamide, acetates, and/or combinations thereof. According to some embodiments, a solvent is ethanol. A solvent is isobutanol in some embodiments. According to some embodiments, two or more pharmaceutical agents may be dissolved or dispersed in the same solvent. For example, dexamethasone, estradiol, and paclitaxel may be dissolved in isobutanol. Alternatively, dexamethasone, estradiol, and paclitaxel may be dissolved in ethanol. In yet another example, dexamethasone, estradiol, and ABT-578, i.e., the rapamycin analog, 3S,6R,7E,9R,10R,12R,14S,15E,17E,19E,21S,23S,26R,27R,34aS)9,10,12,13,14,21,22,23,24, 25,26,27,32,33,34,34a-Hexadecahydro-9,27-dihydroxy-3-[(1R)-2-[(1S,3R,4R)-3-methoxy-4-tetrazol-1-yl)cyclohexyl]-1-methylethyl]-10,21-dimethoxy-6,8,12,14,20,26-hexamethyl-2-3,27-epoxy-3H-pyrido[2,1-c][1,4]oxaazacyclohentriacontine-1,5,11,28,29(4H,6H,31H)-pentone; 23,27-Epoxy-3H-pyrido[2,1-c][1,4]oxaazacyclohentriacontin-e-1,5,11,28,29(4H,6H,31H)-pentone, may be dissolved together in one solvent (e.g., ethanol or isobutanol).

According to some embodiments of the disclosure, a pharmaceutical agent may be a gene therapy agent. For example, a pharmaceutical agent may include a viral or retroviral vector (e.g., adenovirus) having a therapeutic nucleic acid (e.g., a sense or antisense sequence). A pharmaceutical agent may include, for example, a small interfering RNA (siRNA). A siRNA may include a 21 base pair double stranded RNA and may, for example, reduce production of BMP's (e.g., to prevent spinal fusion) or reduce production of cytokines and/or other proteins (e.g., to reduce inflammation and/or promote healing). A siRNA may be complexed with a transfection agent or carrier.

### Systems

A system may include, according to some embodiments, a first spinal implant having a smooth surface coated with a surface composition comprising a pharmaceutical agent and a second spinal implant. A second implant may or may not have a smooth surface coated with a surface composition comprising a pharmaceutical agent. The implant bodies, surface compositions, and/or pharmaceutical agents of the first and second implants may be the same or different. For example, both may be bone screws. In another example, one may be a bone screw and one may be a bone plate.

In some embodiments, a system may include a first pharmaceutical agent formulated for delivery (e.g., intravenous delivery, intramuscular delivery, oral delivery, transdermal delivery, and the like) and a spinal implant having a smooth surface coated with a surface composition comprising a second pharmaceutical agent. A first and second pharmaceutical agent may be the same or different.

### Methods

A method of preparing a spinal implant having an implant and at least one coated surface may include, according to some embodiments, contacting a bare surface of an implant with a surface composition (e.g., a lubricant polymer) to form a coating thereon. In some embodiments, a method may further comprise mixing a surface composition with a pharmaceutical agent (e.g., prior to contacting the implant).

A mixture, in some embodiments, may be applied to a surface (e.g., a bare surface) of an implant by spraying, dipping, jetting and/or any other application techniques. According to some embodiments, at least one polymer may be a crosslinkable polymer (e.g., phosphorylcholine-linked methacrylate polymer). The at least one polymer may include a trimethoxysilane functional group in some embodiments. The at least one polymer and at least one pharmaceutical agent may be mixed using ethanol as a solvent. A mixture may be uniformly applied to at least a portion of an implant surface. Also, the at least one pharmaceutical agent may be uniformly distributed in the coating, layered or otherwise disbursed or dissolved in or on the coating or coatings. A surface composition may have a thickness of from about 0.1 microns to about 1 millimeter, from about 0.1 microns to about 10 microns, from about 0.5 microns to about 50 microns, from about 1 micron to about 10 microns, from about 1 micron to about 100 microns, from about 5 microns to about 500 microns, and/or from about 10 microns to about 1 millimeter.

Depositing a surface composition (e.g., lubricant polymer) on an implant surface may comprise applying, loading, coating, spraying, covering, injecting, printing, depositing, and/or otherwise placing a material on or in an implant (e.g., on a surface and/or in a well). For example, an implant may be dip-coated with a material and excess material optionally may be removed (e.g., wiped, washed) from the surface. In some embodiments, a surface composition may be deposited on a surface by vacuum deposition. A surface composition may be deposited on a spinal implant surface by printing (e.g., using a 3-D ink-jet printer) according to some embodiments. For example, a method of manufacturing a spinal implant may include (a) providing a fluid-dispenser (e.g., a fluid-jetting device) having a dispensing element operable to dispense a material (e.g., a surface composition) in discrete droplets, wherein each droplet has a controlled trajectory, (b) creating relative movement between the dispensing element and the implant (e.g., prosthesis) to define a dispensing path, and (c) selectively dispensing the material from the fluid-dispenser in a raster format to a predetermined portion of the spinal implant along the dispensing path. In some embodiments, a material may be selectively dispensed from a dispensing element to a predetermined portion of a spinal implant in a raster format along a dispensing path. In some embodiments, a surface composition may be deposited in a recess in one or more passes by a fluid dispenser (e.g., ink-jet printer head). The thickness and/or amount of material in a coating may be controlled, in some embodiments, by the number of passes and/or the amount of material dispensed (e.g., drop size).

A raster format may be a continuous or non-continuous dispensing pattern of droplets of material dispensed at specific intervals. The relative motion of the dispensing element and the spinal implant to be loaded with beneficial agent creates a dispensing path which includes a sequential series of linear parallel passes that traverse back and forth along one axis of the spinal implant. The relative motion may be continued in a linear manner between forward and backward or right to left and left to right or upward and downward, depending on the frame of reference. A traversal or a pass is completed when the relative motion reverses direction. That is, relative motion continues past the spinal implant, and then decelerates, stops, reverses direction and accelerates to a constant velocity. After each pass, the position of the dispensing element or spinal implant relative to the dispensing element may be changed or incremented such that additional droplets do not impact in the same location during the subsequent pass. In some embodiments, some overlap may be permitted. A fluid dispenser may be used in combination with a detector operable to detect the location of a recess and a controller that is operable to receive input from the detector and direct the position and/or material output of the fluid dispenser.

A method, according to some embodiments, may include curing a surface composition (e.g., to make the surface coating lubricous). Curing a surface composition may include hydrating, heating, and/or irradiating the surface composition, either independently or by way of another processing step in the overall manufacture of a device (e.g., spinal implant). In some embodiments, a method may comprise applying a surface composition comprising two or more layers of material. Each layer of material independently may have the same or different composition from other layers in the surface composition. A method may include applying a base layer, according to some embodiments. A base layer may adhere, for example, a lubricant polymer to a device (e.g., a spinal implant surface). A method may include, according to some embodiments, applying an overcoating to at least a portion of a device (e.g., a spinal implant surface).

According to some embodiments, a device may be sterilized (e.g., by irradiation) before and/or after application of a surface composition. Prior to being sterilized, a coated surface may be cured, dried, and/or otherwise processed in accordance with a desired end product. According to some embodiments, a sterilizing step may facilitate crosslinking of a polymer coating. A sterilizing step may include exposing a coated implant to at least one cycle of ethylene oxide and/or heat.

A coated device, in some embodiments, may include at least one pharmaceutical agent. For example, a coated scaffold may include about 10 to about 13 micrograms of a pharmaceutical agent along a linear millimeter of a coated implant length or as needed to obtain an effective tissue concentration for the required length of time, for the desired end product.

Any dose that leads to a desired or required effective tissue concentration may be used in some embodiments. Effective tissue concentration limits may be known for many drugs. In some such cases, it may be possible to predict the effective tissue concentration when the drug is released from a device. In others, routine dosing experiments may be performed to determine the right dose or desired dose. Concentration of a drug in the tissue may vary with distance from the device and/or may vary in relation to fluid dynamics near the device, e.g., (lymphatic) drainage,

In some embodiments, an implant of the present disclosure may include a pharmaceutical agent in any amount desired by a practitioner. One of ordinary skill in the art having the benefit of the present disclosure understands that the exact selection and dose of a pharmaceutical depends on a variety of factors including without limitation, one or more aspects of a subject's medical history (e.g., health, allergies, weight), the intended location of the scaffold, the condition being treated, and the intended course of therapy. An implant may include a certain weight of pharmaceutically active agent per unit surface area of device placed in contact with the tissue of interest in order to obtain an effective tissue concentration for a required and/or desired time. For example, an implant may include from about 0.01 micrograms to about 10 milligrams of a pharmaceutical agent along a linear millimeter of a surface and/or its total length. For example, an implant may include from about 0.01 micrograms to about 0.1 micrograms, from about 0.1 micrograms to about 1.0 micrograms, from about 1.0 micrograms to about 10 micrograms, from about 10 micrograms to about 100 micrograms, from about 100 micrograms to about 1.0 milligram, and/or from about 1.0 milligram to about 10 milligrams of a pharmaceutical agent along a linear millimeter of a surface and/or its total length. In some embodiments, these ranges may apply to an implant that includes a pharmaceutical agent in its fibers (e.g., rather than as a coating) and/or in domains.

A coated scaffold may include 30% by weight of a therapeutic agent relative to the polymer or as needed for the desired end product. An implant, according to some embodiments, may include a pharmaceutical agent in any amount relative to the weight of the surface composition desired by a practitioner. For example, an implant may include from about 0.01% by weight to about 0.1% by weight, from about 0.1% by weight to about 1.0% by weight, from about 1.0% by weight to about 10% by weight, from about 1.0% by weight to about 10% by weight, from about 10% by weight to about 20% by weight, from about 20% by weight to about 30% by weight, from about 30% by weight to about 40% by weight, from about 40% by weight to about 50% by weight, and/or more than about 50% by weight of a pharmaceutical agent.

As will be understood by those skilled in the art who have the benefit of the instant disclosure, other equivalent or alternative devices, systems, and methods for reducing an inflammatory response, reducing friction, and/or delivering a pharmaceutical compound to a spine can be envisioned without departing from the essential characteristics thereof. Accordingly, the manner of carrying out the disclosure as shown and described is to be construed as illustrative only.

Persons skilled in the art may make various changes in the shape, size, number, and/or arrangement of parts without departing from the scope of the instant disclosure. For example, a surface composition may be proportioned such that it does not compromise the structural integrity and/or structural function of an implant. Also, where numerical values and/or ranges have been provided, the disclosed values/endpoints may be treated as exact and/or estimates as desired or demanded by the particular embodiment In addition, it may be desirable in some embodiments to mix and match range endpoints. A surface composition (e.g., comprising a pharmaceutical compound) may be deposited on an implant by any available method. In addition, a biocompatible and/or biodegradable material may be deposited on an implant and/or mixed into a surface composition by any available method. For example, a biodegradable material may be applied, printed, and/or coated (e.g., sprayed or spray-dried) onto an implant. These equivalents and alternatives along with obvious changes and modifications are intended to be included within the scope of the present disclosure. Accordingly, the foregoing disclosure is intended to be illustrative, but not limiting, of the scope of the disclosure as illustrated by the following claims.

What is claimed is:

1. A spinal implant, said implant comprising:
a spinal implant body having a surface; and
a surface composition covering at least a portion of the surface, the surface composition comprising a biocompatible lubricant polymer and a pharmaceutically effective amount of a pharmaceutical compound.

2. A spinal implant according to claim 1, wherein the spinal implant body comprises a pedicle screw, a polyaxial screw, an interbody spacer, an anterior cervical plate, an open hook, a rod, a rod-to-rod connector, a cable, a cam, a bone plate, a bone screw, a vertical endplate, a cage, an artificial disc, and combinations thereof.

3. A spinal implant according to claim 1, wherein the spinal implant comprises a material selected from the group consisting of bioglass, a blood cell, a bone—allograft, a bone autograft, a bone cement, a bone chip, calcium, calcium carbonate, calcium phosphate, calcium sulfate, a ceramic, a demineralized bone, a glass, gold, a liposome, a mesenchymal cell, an osteoblast, a phosphate glass, a platelet, albumin, casein, a whey protein, a plant protein, a fish protein), a steel, a synthetic cancellous bone void filler, a thrombin, titanium, tricalcium phosphate, trimethylene carbonate (TMC), and combinations thereof.

4. A spinal implant according to claim 1, wherein the biocompatible lubricant polymer comprises a material selected from the group consisting of a photo polymerizable semi-interpenetrating anhydride network, a crosslinked polymer network with an associated long chain, biocompatible hydrophilic polymer, a hyaluronan hydrogel, a poly(ortho ester), a high molecular weight dextran polymer, high density polyethylene, low density polyethylene, polytetrafluoroethylene, and combinations thereof.

5. A spinal implant according to claim 4, wherein the biocompatible lubricant polymer consists of a photo polymerizable semi-interpenetrating anhydride network comprising a photo polymerizable monomer selected from the group consisting of a methacrylic anhydride of sebacic acid (SA-Me₂), a methacrylic anhydride of 1,3bis(p-carboxy phenoxy)propane (CPP-Me₂), and combinations thereof.

6. A spinal implant according to claim 4, wherein the biocompatible lubricant polymer consists of a crosslinked polymer network and an associated long chain, biocompatible hydrophilic polymer, the crosslinked polymer network comprising a polymer selected from the group consisting of a polyacrylate, a polymethacrylate, a polyurethane, a polyethylene and polypropylene co-difunctional polymer, a polyvinyl chloride, a epoxide, a polyamide, a polyester and alkyd copolymer, and combinations thereof.

7. A spinal implant according to claim 4, wherein the biocompatible lubricant polymer consists of a crosslinked polymer network and an associated long chain, biocompatible hydrophilic polymer, the associated long chain, biocompatible hydrophilic polymer comprising a polymer selected from a poly(N-vinyl lactam, a poly(vinylpyrrolidone), a poly(ethylene oxide), a poly(propylene oxide). a polyacrylamide, a cellulosic material, methyl cellulose, a polyanhydride, a polyacrylic acid, a polyvinyl alcohol, a polyvinyl ether, and combinations thereof.

8. A spinal implant according to claim 4, wherein the hyaluronan hydrogel comprises crosslinked hyaluronan-adipic dyhydrazide and hyaluronan-aldehyde.

9. A spinal implant according to claim 4, wherein the poly(ortho ester) is selected from the group consisting of poly(ortho ester) I, poly(ortho ester) II, poly(ortho ester) III, poly(ortho ester) IV, and combinations thereof.

10. A spinal implant according to claim 4, wherein the high molecular weight dextran polymer comprises dextran having a molecular weight from about 100,000 daltons to about 2,000,000 daltons.

11. A spinal implant according to claim 1, wherein the pharmaceutical compound comprises a compound selected from the group consisting of an adhesive, an arterial vessel wall irritant, a bone morphogenic protein, an extracellular matrix component, an inflammatory cytokine, a polymer, and combinations thereof.

12. A spinal implant according to claim 1, wherein the pharmaceutical compound comprises a compound selected from the group consisting of an analgesic, an antimicrobial agent, an anti-inflammatory agent, a fibrosis-inducing agent, and combinations thereof.

13. A spinal implant according to claim 1, wherein the surface is from about 0.5 millimeters to about 20 millimeters in its longest dimension.

14. A spinal implant according to claim 1, wherein the surface has a surface roughness (Rₐ) of from about 25 to about 50 measured without the surface composition.

15. A spinal implant according to claim 1, wherein the surface has a surface roughness (Rₐ) of from about 30 to about 45 measured with the surface composition.

16. A spinal implant according to claim 1, wherein the surface has a coefficient of friction of from about 0.001 to about 0.2 measured with the surface composition.

17. A spinal implant according to claim 1, wherein the surface is from about 0.1 microns to about 1 millimeter thick.

18. A method of reducing and/or eliminating an inflammatory response comprising:
contacting at least a portion of a subject's spine with a spinal implant comprising a spinal implant body having a surface, and a surface composition covering at least a portion of the surface, the surface composition comprising a biocompatible lubricant polymer and a pharmaceutically effective amount of a pharmaceutical compound.

19. A method of reducing and/or eliminating an inflammatory response according to claim 18, wherein the spinal implant body comprises a pedicle screw, a polyaxial screw, an interbody spacer, an anterior cervical plate, an open hook, a rod, a rod-to-rod connector, a cable, a cam, a bone plate, a bone screw, a vertical endplate, a cage, an artificial disc, and combinations thereof.

20. A method of reducing and/or eliminating an inflammatory response according to claim 18, wherein the spinal implant comprises a material selected from the group consisting of bioglass, a blood cell, a bone—allograft, a bone autograft, a bone cement, a bone chip, calcium, calcium carbonate, calcium phosphate, calcium sulfate, a ceramic, a demineralized bone, a glass, gold, a liposome, a mesenchymal cell, an osteoblast, a phosphate glass, a platelet, albumin, casein, a whey protein, a plant protein, a fish protein), a steel, a synthetic cancellous bone void filler, a thrombin, titanium, tricalcium phosphate, trimethylene carbonate (TMC), and combinations thereof.

21. A method of reducing and/or eliminating an inflammatory response according to claim 18, wherein the biocompatible lubricant polymer comprises a material selected from the group consisting of a photo polymerizable semi-interpenetrating anhydride network, a crosslinked polymer network with an associated long chain, biocompatible hydrophilic polymer, a hyaluronan hydrogel, a poly(ortho ester), a high molecular weight dextran polymer, a high density polyethylene, a low density polyethylene, a polytetrafluoroethylene, and combinations thereof.

22. A method of reducing and/or eliminating an inflammatory response according to claim 18, wherein the pharmaceutical compound comprises a compound selected from the group consisting of an adhesive, an arterial vessel wall irritant, a bone morphogenic protein, an extracellular matrix component, an inflammatory cytokine, a polymer, and combinations thereof.

23. A method of reducing and/or eliminating an inflammatory response according to claim 18, wherein the pharmaceutical compound comprises a compound selected from the group consisting of an analgesic, an antimicrobial agent, an anti-inflammatory agent, a fibrosis-inducing agent, and combinations thereof.

24. A method of reducing and/or eliminating an inflammatory response according to claim 18, wherein the surface is from about 0.5 millimeters to about 20 millimeters in its longest dimension.

25. A method of reducing and/or eliminating an inflammatory response according to claim 18, wherein the surface has a surface roughness (Rₐ) of from about 25 to about 50 measured without the surface composition.

26. A method of reducing and/or eliminating an inflammatory response according to claim 18, wherein the surface has a surface roughness (Rₐ) of from about 30 to about 45 measured with the surface composition.

27. A method of reducing and/or eliminating an inflammatory response according to claim 18, wherein the surface has a coefficient of friction of from about 0.001 to about 0.2 measured with the surface composition.

28. A method of reducing and/or eliminating an inflammatory response according to claim 18, wherein the surface is from about 0.1 microns to about 1 millimeter thick.

29. A method of manufacturing a spinal implant, said method comprising:
providing a spinal implant body having a smooth surface; and
depositing on the smooth surface a surface composition comprising a biocompatible lubricant polymer and a pharmaceutical compound, wherein the surface composition is configured and arranged to deliver a pharmaceutically effective amount of the pharmaceutical compound to the subject's spine.

30. A method of manufacturing a spinal implant according to claim 29, wherein the depositing further comprises uniformly depositing the surface composition on to the smooth surface.

31. A method of manufacturing a spinal implant according to claim 29, wherein the depositing further comprises applying, loading, coating, covering, injecting, spraying, dipping, or jetting the surface composition on to the smooth surface.

32. A method of manufacturing a spinal implant according to claim 29, wherein the depositing further comprises printing the surface composition on to the smooth surface.

33. A method of manufacturing a spinal implant according to claim 32, wherein the printing further comprises:
(a) providing a fluid-dispenser having a dispensing element operable to dispense the surface composition in discrete droplets, wherein each droplet has a controlled trajectory;
(b) creating relative movement between the dispensing element and the implant (e.g., prosthesis) to define a dispensing path; and
(c) selectively dispensing the surface composition from the fluid-dispenser in a raster format to a predetermined portion of the spinal implant surface along the dispensing path.

34. A method of manufacturing a spinal implant according to claim 29, wherein the depositing further comprises depositing a sufficient amount of the surface composition to give from about 1 microgram to about 100 micrograms of the pharmaceutical agent per millimeter of the surface.

35. A method of manufacturing a spinal implant according to claim 29 further comprising curing the surface composition.

36. A method of manufacturing a spinal implant according to claim 35, wherein the curing further comprises hydrating, heating, or irradiating the surface composition.

37. A method of manufacturing a spinal implant according to claim 29 further comprising sterilizing and/or sanitizing the spinal implant.

38. A system for reducing and/or eliminating an inflammatory response comprising:
a first spinal implant comprising a spinal implant body having a first surface, and a first surface composition covering at least a portion of the first surface, the first surface composition comprising a first biocompatible lubricant polymer and a pharmaceutically effective amount of a first pharmaceutical compound; and
a second spinal implant.

39. A system for reducing and/or eliminating an inflammatory response according to claim 38, wherein the first and second spinal implants are the same.

40. A system for reducing and/or eliminating an inflammatory response according to claim 38, wherein the first and second spinal implants are different.

41. A system for reducing and/or eliminating an inflammatory response according to claim 38, wherein the second spinal implant further comprises a spinal implant body having a second surface, and a second surface composition covering at least a portion of the second surface, the second surface composition comprising a second biocompatible lubricant polymer and a pharmaceutically effective amount of a second pharmaceutical compound.

42. A system for reducing and/or eliminating an inflammatory response comprising:
a pharmaceutical composition comprising a pharmaceutically effective amount of a first pharmaceutical agent and a carrier; and
a spinal implant comprising a spinal implant body having a surface, and a surface composition covering at least a portion of the surface, the surface composition comprising a biocompatible lubricant polymer and a pharmaceutically effective amount of a second pharmaceutical compound.

43. A system for reducing and/or eliminating an inflammatory response according to claim 42, wherein the pharmaceutical composition is configured and arranged for intravenous delivery, intramuscular delivery, oral delivery, or transdermal delivery to subject.

44. A system for reducing and/or eliminating an inflammatory response according to claim 42, wherein the first and second pharmaceutical agents are the same.

45. A system for reducing and/or eliminating an inflammatory response according to claim 42, wherein the first and second pharmaceutical agents are different.

46. An implantable bone screw comprising:
an implantable bone screw body having bone screw threads and a bone screw head having a surface; and
a surface composition covering at least a portion of the surface, the surface composition comprising a biocompatible lubricant polymer and a pharmaceutically effective amount of a pharmaceutical compound.

47. An implantable bone screw according to claim 46, wherein the bone screw is configured and arranged as a pedicle screw or a polyaxial screw.

48. An implantable bone plate comprising:
an implantable bone plate body having a surface; and
a surface composition covering at least a portion of the surface, the surface composition comprising a biocompatible lubricant polymer and a pharmaceutically effective amount of a pharmaceutical compound.

49. An implantable bone plate according to claim 48, wherein the bone plate is configured and arranged as a cervical plate.
